(19) United States
(12) Patent Application Publication
(10) Pub. No.: US 2025/0152936 A1
(43) Pub. Date: May 15, 2025

(54) METHOD FOR THE TREATMENT OF VASCULAR ANOMALIES BY ADMINISTERING ONE OR MORE SCLEROSING AGENTS AND APPLYING A SERIES OF ELECTRIC PULSES

(71) Applicant: IGEA S.P.A., Carpi (IT)

(72) Inventors: Ruggero Cadossi, Carpi (IT); Matteo Cadossi, Carpi (IT); Alessandro Zanasi, Carpi (IT); Donata Marazzi, Carpi (IT)

(21) Appl. No.: 18/388,643

(22) Filed: Nov. 10, 2023

(51) Int. Cl. A61N 1/32; A61K 9/00; A61K 31/08; A61K 38/14; A61K 41/00; A61N 1/04; A61N 1/36; A61P 9/14

(52) U.S. Cl. CPC A61N 1/327; A61K 9/0019; A61K 31/08; A61K 38/14; A61K 41/0047; A61N 1/0412; A61N 1/36034; A61P 9/14

(57) ABSTRACT

A method for treatment of a vascular anomaly can include administering one or more sclerosing agents in a subject in need thereof, and using electric pulses to increase the permeability of cell walls.

### BACKGROUND

### Field of the Disclosure

The present invention relates to a method for treatment of a vascular anomaly in a subject in need thereof, in particular using electric pulses to increase the permeability of cell walls, and, more specifically to provide access of drugs directly into the cell cytosol.

### Brief Description of Related Technology

The effectiveness of many drugs is limited by the inability of the drug to penetrate the cell membrane. Although the dosage of the drug may be increased in order for the drug to have its desired effect on the target cell, such increases in dosages often result in significant negative side effects. The need exists, therefore, for a method of introducing drugs into the target cell while at the same time minimizing the death of healthy cells.

Electrosclerotherapy is an experimental therapy that involves the application of specific electric pulses to a target cell resulting in the increased permeability of the cell membrane. This increased permeability by electric pulses, known as “electroporation,” allows a greater number of the drug molecules to enter the target cells. This further allows a much lower concentration of the drug to be introduced without sacrificing efficacy and at the same time reducing or eliminating known undesired side effects.

The delivery of electrical pulses induces a local “vascular lock” that maintains a local high concentration of the sclerosing agents and has a vascular disrupting effect on the endothelium itself.

Vascular anomalies, in particular vascular malformations, are rare benign congenital anomalies consisting of dysplastic vascular structures that are often tubular or spongy, not solid and contain blood or lymphatic fluid.

Bleomycin is a drug that is used as a cytostatic in the treatment of cancer and for its sclerosing properties in the treatment of vascular malformations.

On a clinical level, the combination of bleomycin and electroporation in the treatment of skin tumors has been known under the name of electrochemotherapy since the early 1990s. With regard to vascular malformations, the first publication dates back to 2017. Early studies indicated that the use of bleomycin in combination with reversible electroporation can increase the effectiveness of sclerotherapy, even with a reduced dose of drug and a reduced number of sessions compared to standard sclerotherapy with bleomycin. In 2021, a retrospective study of 17 patients with venous malformations that did not respond to previous invasive therapies showed a decrease in lesion volume on magnetic resonance imaging by an average of 86%, with clinical improvement in all patients after an average of 3.7 months and 1.12 sessions per patient.

If not appropriately calibrated, however, the combination of drug and electric pulses in vivo can produce oedema and can cause necrosis of the surrounding tissue. The need exists, therefore, to calibrate the correct amounts of drug as well as the energy, duration and number of electric pulses to be applied with the one or more drugs.

### SUMMARY

In view of the above, it is an object of the present invention to provide a method for the treatment of a vascular anomaly which increases effectiveness of the sclerosing agent/s or reduces local and systemic toxicity or side effects of the sclerosing agent/s, relative to a subject that suffers a vascular anomaly but to whom the effective electrical pulses were not applied.

### BRIEF DESCRIPTION OF THE DRAWINGS

FIGS. 1A and 1C show magnetic resonance of a symptomatic venous malformation of the left thigh in a 10-year-old patient previously treated with surgery and sclerotherapy;

FIGS. 1B and 1D show magnetic resonance of the symptomatic venous malformation of FIG. 1A three months after one electrosclerotherapy session. The magnetic resonance shows complete resolution.

FIG. 2A shows a photograph of a venous malformation after 7 cycles of sclerotherapy with ethanol.

FIG. 2B shows a photograph of the venous malformation of FIG. 2A after two cycles of electrosclerotherapy with bleomycin.

FIG. 2C shows postoperative and long term 3 years follow-up of the venous malformation of FIGS. 2A and 2B.

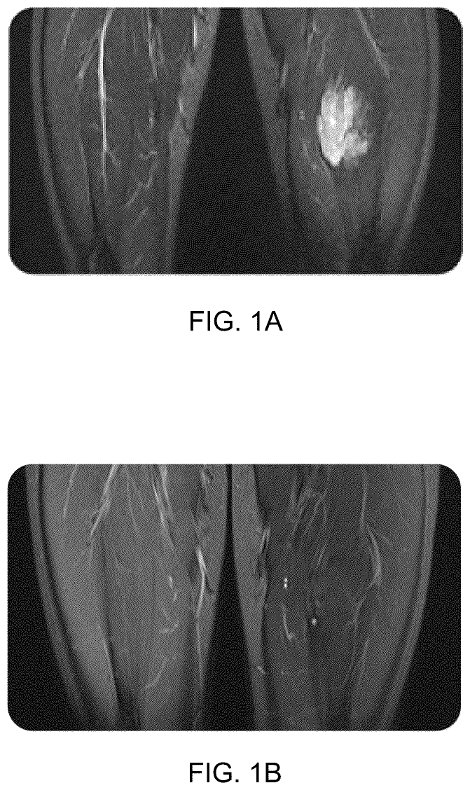

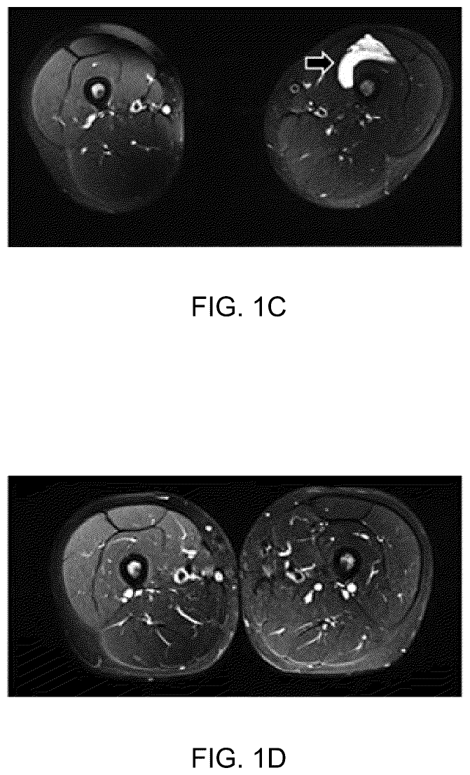

### DETAILED DESCRIPTION

The method for treatment of a vascular anomaly in a subject in need thereof according to the invention comprises:
(a) administering one or more sclerosing agents to the subject in need thereof by systemic or local injection such that the one or more sclerosing agents is/are delivered to the vascular anomaly; and
(b) applying to the subject in need thereof at the site of the vascular anomaly, a series of electric pulses having an energy, defined as number of pulses×voltage of each pulse×duration of pulse, in the range from 15 to 25 Joule for inducing electroporation, wherein the application of pulses increases effectiveness of the sclerosing agent/s or reduces local and systemic toxicity or side effects of the sclerosing agent/s, relative to a subject that suffers a vascular anomaly but to whom the effective electrical pulses were not applied.

The series of electric pulses temporarily increases the permeability of cell membranes, thereby increasing the efficiency of intracellular transport of the one or more sclerosing agents and reducing the dose required to achieve equal efficacy in treating vascular anomalies.

The number of electrical pulses is preferably from 3 to 30 electrical pulses at 500 to 1500 V/cm having pulse duration of 50 to 100 μs.

The sclerosing agent is preferably one or more of ethanol, sodium tetradecyl sulfate (STS), Picibanil (OK 432), polidocanol, doxycycline or bleomycin. More preferably the sclerosing agent is bleomycin.

The subject in need of treatment is preferably human. However, the method of treatment can also be used on animals.

The one or more sclerosing agents may be administered to the subject in need thereof by systemic intravenous injection or by local injection at the site of the vascular anomaly. The amount of sclerosing agent administered is much lower than would be necessary without the application of electric pulses in the case of local injection. In the case of systemic injection, the amount of sclerosing agent is not necessarily lower, but the result of applying electric pulses improves drug effectiveness, induces a vascular lock that keeps the drug confined locally, and disrupts the barrier function of the endothelium.

When administered by systemic injection, the one or more sclerosing agents are administered at a dose of 0.01 to 0.5 mg/Kg of weight of the subject, preferably at a dose of 0.1 to 0.3 mg/Kg of weight of the subject, more preferably at a dose of 0.2 mg/Kg of weight of the subject. When administered by systemic injection, electric pulses are applied preferably within 8 minutes from injection of the sclerosing agent. In an alternative embodiment, electric pulses can be applied before the application of bleomycin and the injection is arterio-venous.

When the one or more sclerosing agents are administered to the subject in need thereof by local injection at the site of the vascular anomaly, the one or more sclerosing agents are administered at a maximum dose of 0.01 to 5 mg/Kg of weight of the subject, more preferably at a dose of 0.00125 to 1.25 mg/cm³ of volume of vascular anomaly to be treated, more preferably at a dose of 0.0125 to 1.25 mg/cm³ of volume of vascular anomaly to be treated, even more preferably at a dose of 0.125 mg/cm³ of volume of vascular anomaly to be treated. When administered by local injection at the site of the vascular anomaly, electric pulses are preferably applied after about 1 minute from injection of the sclerosing agent. In an alternative embodiment, electric pulses can be applied before the application of bleomycin and the injection is transarterial.

The sclerosing agents may be administered to the subject in need thereof by transmucosal, transcutaneous or endovascular injection.

In a preferred embodiment, the series of electrical pulses is of 1 to 10 electrical pulses, preferably of 8 electrical pulses. The electrical pulses are preferably at 600 to 1200 V/cm more preferably at 1000 V/cm.

The duration of the electrical pulses is preferably from 50 to 100 μs.

The electrical pulses may be applied by fixed or custom geometry electrodes. In addition, the electric pulses may be applied by partially insulated electrodes, so as to protect the surrounding tissue and avoid necrosis.

In a preferred embodiment the site of a vascular anomaly is covered by the treatment from a volume of 50% of the vascular anomaly to a volume of 100% of the vascular anomaly. Ideally, the higher percentage of volume treated, the greater the efficacy of treatment. However this must be evaluated in cases in which the anomaly covers a large area, due to possible countereffects of extensive therapy.

The vascular anomaly is preferably a vascular malformation.

Vascular malformations are rare benign congenital anomalies consisting of dysplastic vascular structures that are often tubular or spongy, not solid and contain blood or lymphatic fluid. Vascular malformations may be low-flow or high-flow, depending on whether the arterial component is absent or present, respectively, and occur mainly in the head and neck region.

The most frequent low-flow vascular malformations are, in order, venous and lymphatic malformations.

Venous malformations (VM) consist of dilated and spongy venous channels of varying size. Histologically they have abnormally developed venous walls with defects in the smooth muscle layer and absence of valves. Most VMs become symptomatic due to blood stagnation and localized thrombosis/thrombolysis. VM may at the same time cause disfiguration or impairment of neighboring structures and organs (e.g. airway obstruction). In summary, many patients with VM suffer and have an overall reduced physical function.

Lymphatic malformations (LM) consist of cystic spaces filled with lymph fluid and dilated lymphatic channels. These spaces can be macrocystic or microcystic depending on their size. Indications for invasive therapy of LMs are usually recurrent infections, recurrent haemorrhage, impairment of neighbouring structures (e.g. upper airways) and disfiguration.

Arteriovenous malformations are defined as abnormal connections between arteries and veins without an intermediate capillary bed. In advanced stages, patients develop venous hypertension, haemorrhage or tissue ischaemia/necrosis near the lesion. AVMs usually become progressively symptomatic over time and if extensive can cause heart failure.

The method according to the invention represents an effective therapy for vascular anomalies, in particular vascular malformations, for which therapy to date is not satisfactory.

### EXAMPLES

### Example 1

A 10-year-old male patient with a symptomatic venous malformation of the left thigh, causing swelling, pain, and partial immobility, recurring after surgery and sclerotherapy with Aethoxysklerol was treated with electrosclerotherapy using custom geometry electrodes and 1.5 mg bleomycin. Magnetic resonance imaging studies at 3 months after one session of electrosclerotherapy show complete resolution of the malformation in T2-weighted short tau inversion recovery coronal and axial magnetic resonance images. Volume of the lesion before treatment: 26.79 cm³; volume of the lesion after treatment: 0.00 cm³. No documented adverse events.

### Example 2

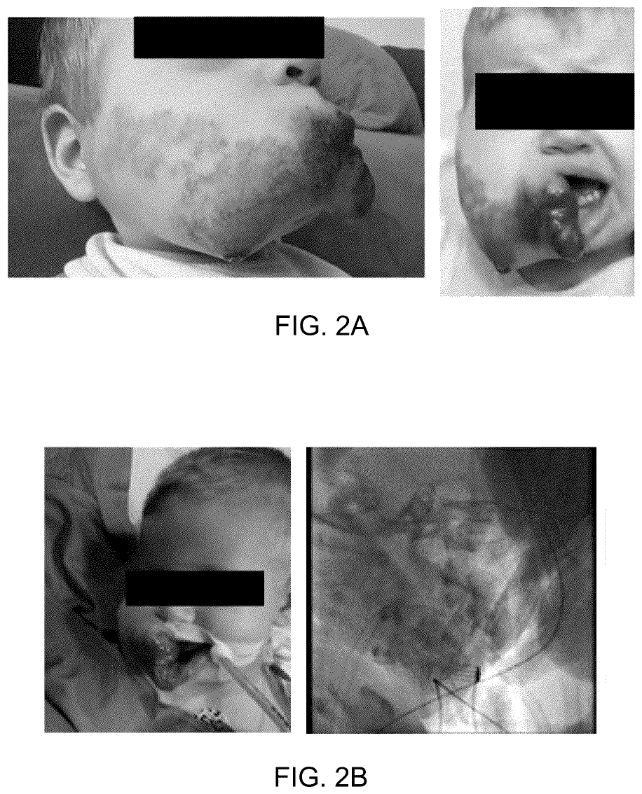

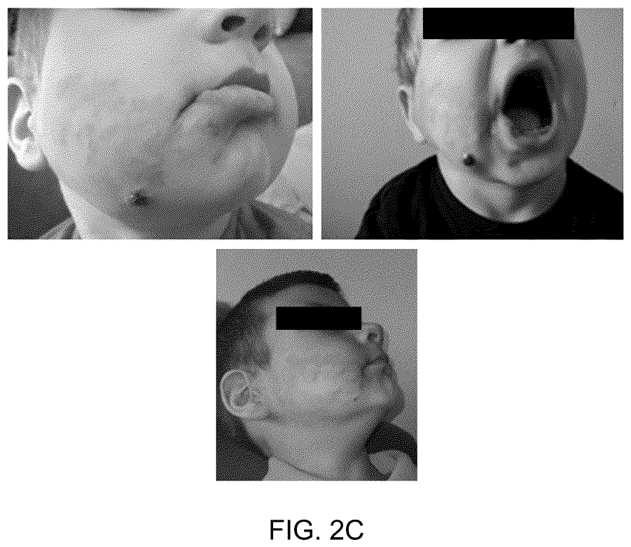

FIG. 2A shows a male patient unsuccessfully treated with 7 sessions of ethanol sclerotherapy.

FIG. 2B shows the same male patient after receiving 2 sessions of electrosclerotherapy with finger electrode and 2 mg of Bleomycin per session.

FIG. 2C shows postoperative and long term 3 years follow-up. Functionality of nerves is preserved.

What is claimed:

1. A method for treatment of a vascular anomaly in a subject in need thereof comprising:
(a) administering one or more sclerosing agents to the subject in need thereof by systemic or local injection such that the one or more sclerosing agents are delivered to the vascular anomaly; and
(b) applying to the subject in need thereof at the site of the vascular anomaly, a series of electric pulses having an energy, defined as number of pulses×voltage of each pulse×duration of pulse, and being in a range from 15 to 25 Joule for inducing electroporation,
wherein the application of pulses is effective to increase effectiveness of the one or more sclerosing agents or to reduce local and systemic toxicity or side effects of the one or more sclerosing agents, relative to a subject that suffers a vascular anomaly but to whom the effective electrical pulses were not applied.

2. The method of claim 1, wherein the number of electrical pulses is from 3 to 30 electrical pulses at 500 to 1500 V/cm having pulse duration of 50 to 100 μs.

3. The method of claim 1, wherein the sclerosing agent is one or more of ethanol, sodium tetradecyl sulfate (STS), Picibanil (OK 432), polidocanol, doxycycline, and bleomycin.

4. The method of claim 3, wherein the sclerosing agent is bleomycin.

5. The method of claim 1, wherein the subject is human.

6. The method of claim 1, wherein the one or more sclerosing agents are administered to the subject in need thereof by systemic injection.

7. The method of claim 6, wherein the one or more sclerosing agents are administered at a dose of 0.01 to 0.5 mg/Kg of weight of the subject.

8. The method of claim 7, wherein the one or more sclerosing agents are administered at a dose of 0.1 to 0.3 mg/Kg of weight of the subject.

9. The method of claim 8, wherein the one or more sclerosing agents are administered at a dose of 0.2 mg/Kg of weight of the subject.

10. The method of claim 1, wherein the one or more sclerosing agents are administered to the subject in need thereof by local injection at the site of the vascular anomaly.

11. The method of claim 10, wherein the one or more sclerosing agents are administered to the subject in need thereof by transmucosal, transcutaneous, or endovascular injection.

12. The method of claim 11, wherein the one or more sclerosing agents are administered at a maximum dose of 0.01 to 5 mg/Kg of weight of the subject.

13. The method of claim 10, wherein the one or more sclerosing agents are administered at a dose of 0.00125 to 1.25 mg/cm³ of volume of vascular anomaly to be treated.

14. The method of claim 13, wherein the one or more sclerosing agents are administered at a dose of 0.125 mg/cm³ of volume of vascular anomaly to be treated.

15. The method of claim 1, wherein the series of electrical pulses is of 1 to 10 electrical pulses.

16. The method of claim 15, wherein the series of electrical pulses is of 8 electrical pulses.

17. The method of claim 1, wherein the electrical pulses are at 600 to 1200 V/cm.

18. The method of claim 17, wherein the electrical pulses are at 1000 V/cm.

19. The method of claim 1, wherein the electrical pulses have a pulse duration of 50 to 100 μs.

20. The method of claim 1, wherein the electrical pulses are applied by fixed or custom geometry electrodes.

21. The method of claim 1, wherein the site of vascular anomaly is covered by the treatment from a volume of 50% of the vascular anomaly to a volume of 100% of the vascular anomaly.

22. The method of claim 1, wherein the vascular anomaly is a vascular malformation.

23. The method of claim 1, wherein the electric pulses are applied by partially insulated electrodes.
